(19) United States
(12) Patent Application Publication
(10) Pub. No.: US 2017/0071468 A1
(43) Pub. Date: Mar. 16, 2017

(54) MOTION TRACKING METHOD FOR SONOGRAPHER

(71) Applicant: Carestream Health, Inc., Rochester, NY (US)

(72) Inventors: Lynn M. La Pietra, Rochester, NY (US); Ajay Anand, Rochester, NY (US); Daniel A. Monaghan, Discovery Bay, CA (US)

(21) Appl. No.: 15/163,792

(22) Filed: May 25, 2016

Related U.S. Application Data
(60) Provisional application No. 62/217,388, filed on Sep. 11, 2015.

(51) Int. Cl. A61B 5/00; A61B 5/107; A61B 8/08; A61B 5/11

(52) U.S. Cl. CPC A61B 5/0004; A61B 5/1116; A61B 5/74; A61B 5/7275; A61B 5/1122; A61B 5/107; A61B 8/5276; A61B 5/4538

(57) ABSTRACT

A method records a biometric signal received from a sensor that is coupled to the skin, clothing, or seating of a sonographer and identifies a pattern from the recorded signal, wherein the pattern relates to sonographer activity for an ultrasound exam. Feedback information about the sonographer activity is provided according to an analysis of the identified pattern.

### CROSS REFERENCE TO RELATED APPLICATIONS

This application claims the benefit of U.S. Provisional application U.S. Ser. No. 62/217,388, provisionally filed on Sep. 11, 2015, entitled “ULTRASOUND METHOD”, in the names of Lynn LaPietra et al., incorporated herein in its entirety.

### TECHNICAL FIELD

The invention relates generally to the field of medical ultrasound systems and methods, and in particular to a method for monitoring activity, movement, and posture of a sonographer.

### BACKGROUND

Ultrasound imaging systems/methods are known, such as those described for example in U.S. Pat. No. 6,705,995 (Poland) and U.S. Pat. No. 5,370,120 (Oppelt) both incorporated herein by reference in their entirety.

A sonographer, or ultrasonographer, is a healthcare professional who specializes in the use of ultrasonic imaging devices to produce diagnostic images, scans, videos, or 3D volumes of anatomy and diagnostic data. The sonographer is often a radiographer, but may be any healthcare professional with the appropriate training.

Sonographers have a high degree of responsibility in the diagnostic process and the skill level and experience of the sonographer can be significant factors for making effective use of the ultrasound imaging system. Sonography requires a technician having specialized education and skills to view, analyze and modify the scan to optimize the information in the image, and requires some understanding of the underlying ultrasound physics as well as training related to cross sectional anatomy, physiology and pathology. Many countries require that medical sonographers have professional certification; certification requirements for clinical practice as a sonographer vary greatly by country.

According to various studies, occupational injury is not uncommon among sonographers and appears to be increasing. Among possible reasons for increased work-related injury are increased workloads due to skilled labor shortages and lower reimbursement rates; increasingly obese patient populations; poor equipment design, constraining the flexibility of positioning the equipment appropriately for each exam type; and an aging workforce.

One set of factors affecting injury rates relates to work organization. Employees may work long hours without breaks, often enticed with bonuses and incentives to work harder, faster, and longer in order to boost throughput. Insufficient training can also be a problem; with a lack of training for employees in the proper ways to perform their duties or in the proper use of the equipment.

Another set of factors includes delayed reporting or diagnosis of injuries or conditions resulting from sonographer activity, and ineffective or inappropriate injury management.

A further set of factors affecting injury rates relates to the work activities that are part of the routine of the sonographer, with these activities often characterized by requirements for:

repetitive motion;

forceful exertions or strain, such as in the effort needed for applying consistent pressure when probing the transducer scan head along a patient's abdomen or for compressing leg veins as part of the exam procedure;

awkward postures or unnatural positions, commonly experienced when reaching over patients during bedside exams, for example;

uncomfortable positioning of limbs, such as flexion, extension or deviation of the hand, wrist, elbows, or shoulders, holding uncomfortably strained positions which may need to be sustained for more than a few seconds for some exam types;

high workload, generally the result of staff downsizing and an increase in the number of exams performed by a technician per day;

movement patterns that involve frequent reaching above shoulder level; and

work activities to which the individual is unfamiliar.

Factors such as these can cause problems such as Musculoskeletal Disorder, or MSD, referring to a group of disorders caused by or aggravated by workplace activities. These neuromusculoskeletal disorders are referred to by a number of names, such as musculoskeletal injury (MSI) and repetitive strain injury (RSI). Although MSD has been described for years in a number of other professions, it has only recently been identified in sonographers.

The problem of occupational injury has become more pronounced with the advent of highly portable ultrasound apparatus that can be moved for bedside use. Using such a portable system, the sonographer may have less control over the imaging environment and may encounter a higher percentage of situations which require special considerations for navigating about the patient and around other equipment, such as life support apparatus, for example.

Accordingly, there is a desire to provide tools and techniques that can help to prevent and reduce the frequency of occupational injuries among sonographers.

### SUMMARY

Certain embodiments described herein address the need for a method for recording and characterizing activity patterns of a sonographer. Embodiments of the present disclosure obtain biometric measurements and analyze the acquired measurements to help determine how to provide feedback to the sonographer for reducing the likelihood of work-related pain or injury.

According to at least one aspect of the invention, there is described a method comprising: recording a biometric signal received from a sensor that is coupled to the skin, clothing, or seating of a sonographer; identifying a pattern from the recorded signal, wherein the pattern relates to sonographer activity for an ultrasound exam; and providing feedback information about the sonographer activity according to an analysis of the identified pattern.

These aspects are given only by way of illustrative example, and such objects may be exemplary of one or more embodiments of the invention. Other desirable objectives and advantages inherently achieved by the disclosed invention may occur or become apparent to those skilled in the art. The invention is defined by the appended claims.

### BRIEF DESCRIPTION OF THE DRAWINGS

The foregoing and other objects, features, and advantages of the invention will be apparent from the following more particular description of the embodiments of the invention, as illustrated in the accompanying drawings. The elements of the drawings are not necessarily to scale relative to each other.

FIG. 1 is a schematic diagram that shows a system for acquiring ultrasound data along with motion data from the sonographer.

FIGS. 2A and 2B are schematic diagrams that show the use of sensors to detect reaching and overhead extension movement patterns.

FIG. 2C is a schematic diagram that shows an alternate sensor configuration in which additional sensors are coupled to supporting furniture used by the sonographer.

FIG. 3 is a logic flow diagram that shows a process for detecting and analyzing movement patterns according to an embodiment of the present disclosure.

FIG. 4 is a plan view of a display arrangement for results reporting.

### DETAILED DESCRIPTION OF THE EMBODIMENTS

The following is a detailed description of embodiments of the invention, reference being made to the drawings in which the same reference numerals identify the same elements of structure in each of the several figures.

Where they are used in the context of the present disclosure, the terms “first”, “second”, and so on, do not necessarily denote any ordinal, sequential, or priority relation, but are simply used to more clearly distinguish one step, element, or set of elements from another, unless specified otherwise.

As used herein, the term “energizable” relates to a device or set of components that perform an indicated function upon receiving power and, optionally, upon receiving an enabling signal.

In the context of the present disclosure, the phrase “in signal communication” indicates that two or more devices and/or components are capable of communicating with each other via signals that travel over some type of signal path. Signal communication may be wired or wireless. The signals may be communication, power, data, or energy signals. The signal paths may include physical, electrical, magnetic, electromagnetic, optical, wired, and/or wireless connections between the first device and/or component and second device and/or component. The signal paths may also include additional devices and/or components between the first device and/or component and second device and/or component.

In the context of the present disclosure, the term “subject” is used to describe the patient that is undergoing ultrasound imaging. The terms “sonographer”, “technician”, and “practitioner” are used to indicate the person who actively operates the sonography equipment.

The term “highlighting” for a displayed element or feature has its conventional meaning as is understood to those skilled in the information and image display arts. In general, highlighting uses some form of localized display enhancement to attract the attention of the viewer. Highlighting a portion of a display, such as a particular value, graph, message, or other element can be achieved in any of a number of ways, including, but not limited to, annotating, displaying a nearby or overlaying symbol, outlining or tracing, display in a different color or at a markedly different intensity or gray scale value than other image or information content, blinking or animation of a portion of a display, or display at larger scale, higher sharpness, or contrast.

In the context of the present disclosure, the term “coupled” is intended to indicate a mechanical association, connection, relation, or linking, between two or more components, such that the disposition of one component affects the spatial disposition of a component to which it is coupled. For mechanical coupling, two components need not be in direct contact, but can be linked through one or more intermediary components. For example, a sensor can be clipped to a shirtsleeve in order to move with the arm of the wearer; thus the sensor can be considered coupled with the corresponding arm of the wearer.

As has been described in the background section of the present application, there is a desire to prevent and reduce the likelihood of occupational injury among sonographers. Such prevention/reduction can be achieved by monitoring movement of the sonographer in a manner that is routine and non-invasive to help to identify patterns of movement that can indicate tendency for work-related injury or pain. Monitoring and reporting movement events and patterns can further help the sonography technician to recover more quickly from injury or discomfort and to avoid relapse, compounding, or further injury.

This can be accomplished by an ergonomic or biometric tracking of a sonographer. The present disclosure provides a method to track sonographer activity, such as monitoring a sonographer's arm and upper body position, and to alert the sonographer to problems such as positioning errors, repetitive motion, awkward and stressful posture, and other measured conditions that can cause stress, discomfort, and possible injury. Biometric tracking can also help to identify related sources of stress, including excessive number of hours without a break, for example. Cumulative tracking of sonographer activity at a particular site can further help to identify staff training needs.

Applicants have developed a method for ergonomically tracking a sonographer to monitor work activities without interfering with carrying out those functions, for the purpose of monitoring/preventing occupational injuries. Using an embodiment of the present disclosure can allow reporting that can help to prevent or mitigate work-related conditions or injury.

Of particular relevance for analyzing injury factors is monitoring sonographer movement in setup and execution of an ultrasound exam. The biometric sensors 30 that are used can measure movement directly or can indirectly measure stress or exertion levels, from which some measure of overall activity can be inferred.

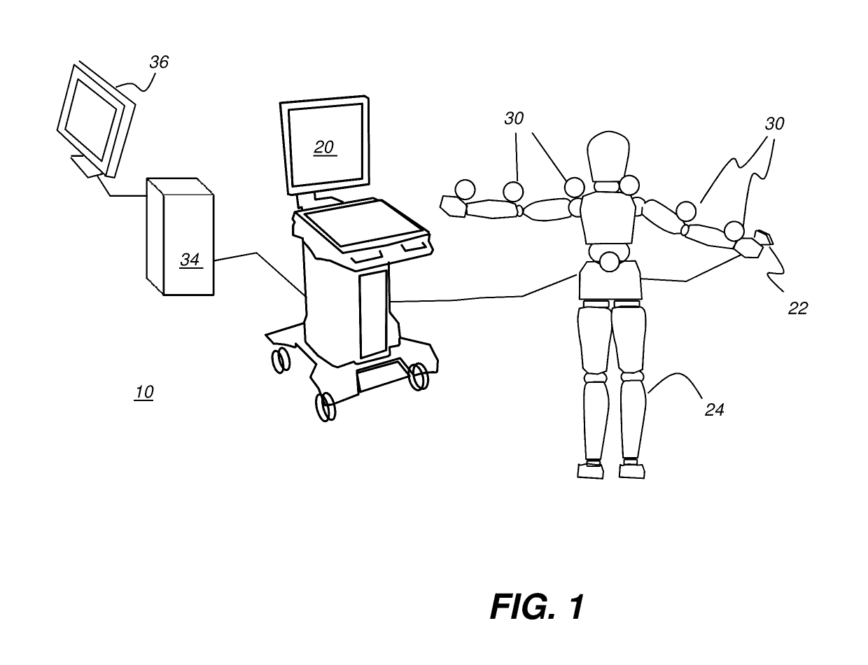

The schematic diagram of FIG. 1 shows an ultrasound apparatus 10 according to an embodiment of the present disclosure. An ultrasound system 20 has a transducer 22 used by a sonographer 24 as a sensing probe for ultrasound imaging. One or more wireless biometric sensors 30 are provided, coupled to the arm and other upper anatomy of the sonographer 24 in some way in order to track movement during image acquisition. Sensors 30 are in signal communication with a processor 34 that can be part of ultrasound system 20 or can be separately provided, such as a networked processor for example. Typically, a wireless connection of sensors 30 is provided. Processor 34 may be in signal communication with ultrasound system 20 or may be used only in order to interact with sensors and other components.

Further, a plurality of sensors 30 can be employed as shown in FIG. 1. Sensors 30 can be of different types, providing a variety of useful data for assessing sonographer activity. A separate display 36 can also be provided, in signal communication with processor 34.

### Movement Analysis

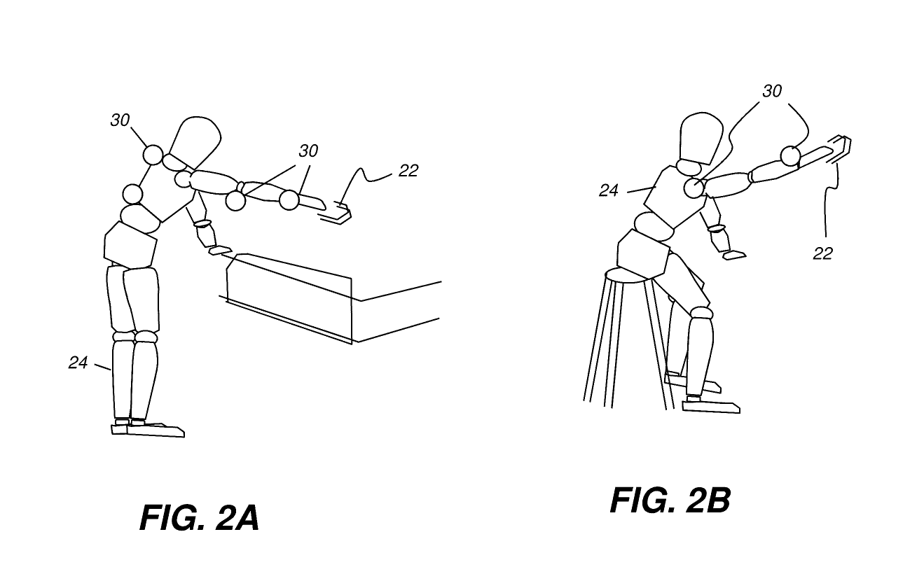

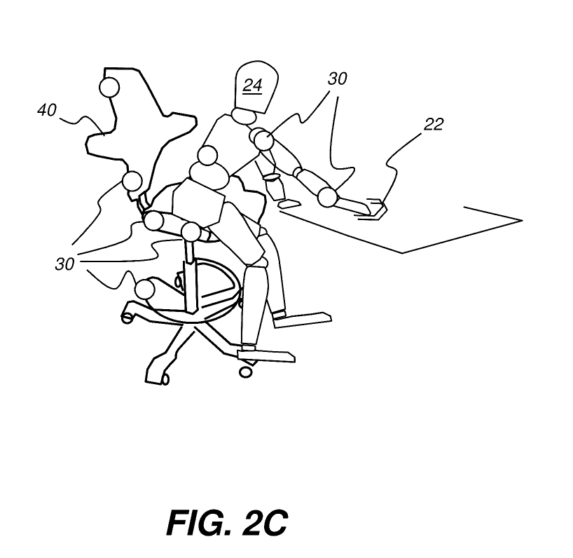

In order to better understand how sensors 30 are used for monitoring sonographer movement, it is instructive to consider a few exemplary movement patterns for which monitoring can be useful. Referring to FIG. 2A, extension of the arm in reaching forward is one example of a movement pattern of interest for pain and injury monitoring. Referring to FIG. 2B, raising of the arm above shoulder level is another movement pattern that can cause problems over time and can be readily detected and tracked. It should be noted that other biometric characteristics of the sonographer 24 can alternately be measured.

In addition to the reaching and overhead extension behavior described with reference to FIGS. 2A and 2B, the apparatus of FIG. 1 allows the collection of data on various other movement patterns and stationary poses that can be of concern relative to sonographer pain and injury. This can include the following movement patterns, for example:

(i) maintaining a stressful position and holding still in such a position for excessive duration;

(ii) pushing, lifting, or pressing inward or outward from an awkward position or exhibiting other forceful exertion or strain;

(iii) poor posture when seated or standing or uncomfortable positioning of limbs;

(iv) repetitive movements;

(v) excessive muscle tension in the shoulders, back, arms; and

(vi) difficult hand or wrist positions, such as positions causing excessive flexion, extension, or deviation of the hand.

The above list is not all-inclusive; there can be any of a number of other movement patterns or stationary positioning patterns that can be of interest for reducing pain and injury levels and that can be detected using one or more sensors 30.

Sensors need not be coupled directly to the sonographer, but may be positioned on supporting equipment. FIG. 2C is a schematic diagram that shows an alternate sensor configuration in which additional sensors are coupled to supporting furniture used by the sonographer. An ergonomically designed chair 40 can allow pivoting to different positions and may support various postures, providing support for the technician when leaning, allowing the sonographer to straddle the chair 40 in a comfortable position, with adjustable settings for height, stiffness, and other factors. Sensors 30 on chair 40 can include weight or pressure sensors or heat sensors for detecting the presence and/or relative position of sonographer 24. Other sensors can include accelerometer and rotational sensors for chair position, for example.

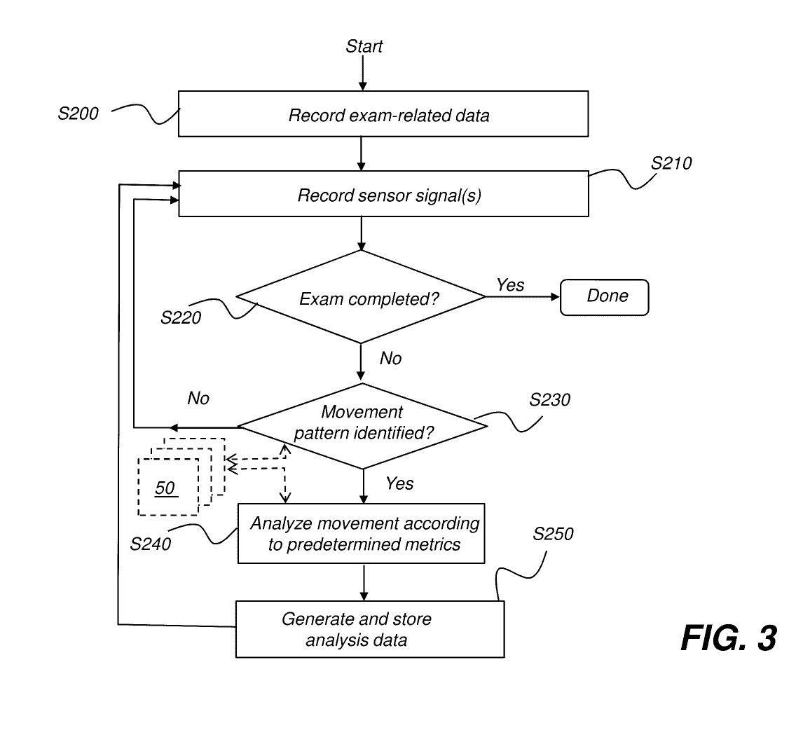

Analysis of movement patterns can compare sensor signals against stored data to determine what type of activity is being performed. Signal characteristics for one or more sensors, such as relative timing and amplitude, can be useful for characterizing a particular pattern of movement, for example. The logic flow diagram of FIG. 3 shows a repeating process for motion analysis that can be executed by processor 34 (FIG. 1) or by processing logic on the ultrasound system 20 itself.

In a recording step S200 in FIG. 3, exam-related data that is relevant to movement characterization is obtained. This can include various types of exam metadata, including date, time, patient ID, facility name, sonographer identification, examination type, and information about the patient, such as metrics for age, sex, height, weight, position (for example: prone, standing, or seated), and other information helpful in movement analysis. Some or all of the exam-related data can be identified and entered to the processor using a scanner, badge reader, or other mechanism that allows selective access to some of the patient data for imaging and treatment professionals.

Sonographer identification can be obtained by a badge scan, password entry, or other mechanism that provides an individualized accounting for each user of the sonography system. According to an embodiment of the present disclosure, each sonographer can have an associated individual profile that can include not only name, employee number, and other identifying data, but also profile information that can be of value in motion assessment. Factors such as weight, height, age, and sex of the sonographer can be helpful for subsequent analysis steps and can be used, for example, to determine how much stress a particular movement pattern may cause. Some positions or movement patterns may be less stressful for a sonographer who is taller than average, or whose height and weight are within a given range. In addition, the profile can include historical information on pain or injury conditions previously reported by the sonographer and relevant to the exertion needed for particular exam types.

Still referring to FIG. 3, in a sensing step S210, the system records signals sequentially received from the sensor(s) 30 that are coupled to the sonographer. A decision step S220 checks to determine if the ultrasound exam is complete and can be terminated, or if the exam continues. A pattern identification step S230 determines, using a database of stored patterns 50 or using other suitable stored information, whether or not the sonographer is executing an identifiable movement pattern. If the pattern can be recognized, a movement analysis step S240 then compares the sequence of sensor signals against corresponding data for known movement patterns. A results storage step S250 then generates and stores the movement analysis data so that it can be reported back to the sonographer.

It can be appreciated that the looping procedure of FIG. 3 can be executed in any of a number of ways and may include variations and other additional steps and procedures, as needed to support a sonographer monitoring at a particular system or facility.

There are a number of ways to provide information for comparison of detected movement from the sensors 30 (FIG. 1) with conventional or target movement patterns. As represented in FIG. 3, the pattern for each type of ultrasound exam can be stored as a type of model. This storage can be performed using conventional logic tools and by recording the measurement activity that generally applies for an idealized patient, with some tolerances provided for how much variability from the preferred movement pattern is permissible. A particular stored pattern 50 can be indexed by information recorded in step S200 (FIG. 3), such as by age, weight, height, sex, and condition of the patient, as well as by the anatomy being examined. One stored pattern 50 can use recorded movement for imaging the lower abdomen of a pediatric patient, age 4; a different stored pattern 50 can record a movement pattern for the same exam type, but for a female, age 70, tall, and moderately obese. Thus, for example, a library of stored patterns 50, indexed by exam type and patient characteristics, can be acquired and updated.

According to an embodiment, machine learning techniques are used to generate and modify sonographer movement patterns that can be used for a particular ultrasound system or medical facility.

Data acquired from sonographer movement can be compared to a database of stored patterns that have been analyzed and classified, such as into categories according to recommended practices and guidelines, such as ideal or best practice, acceptable but not optimal practice, movement patterns or positions not recommended, training required, and hazardous movement or positioning. By correlating the sonographer measured data with the database of stored patterns, a monitoring and analysis system on processor 34 (FIG. 1) can provide user guidance directly to the sonographer or to training and administrative personnel.

Data obtained from one or more sensors can be correlated with the type of ultrasound exam that is being performed. Statistical information generated from analyzing the acquired data can help to identify individual tendencies and trends, as well as problems that may be common for sonographers at a particular site or related to various patient population segments. These results can then be used to improve sonographer ergonomics, adjust case workload, allot equipment resources, and compensate in other ways to help alleviate movement problems.

It is noted that patterns of biometric signals other than signals that are directly indicative of movement can be used. Signals indicating relative body temperature, respiration rate, pressure, and other biometrics can be analyzed and characterized as patterns and used to track the sonographer's work activity.

Data from each sensor can be transmitted/stored to a central server or central display. This transmitted/stored data can then be reviewed at a later time by the sonographer. In addition, this transmitted/stored data can be reviewed by another individual, such as an ergonomic professional or occupational therapist, to provide advice/guidance to the sonographer regarding work activities based on the ultrasound ergonomic metrics. Further, the collection of the transmitted/stored data from a plurality of sonographers can be summarized, as described in more detail subsequently.

Pattern characterization can be performed in any of a number of ways, and pattern recognition can be continually refined for a particular imaging facility or site as well as for a particular individual sonographer. Pattern analysis can consider coarse threshold conditions, such as obvious over-extension, timeouts for holding any extended position, repeated motion, or other measurable conditions. Pattern analysis can alternately rely on training modes for best practices, using a stored, model set of movements executed by a trained sonographer under expert guidance.

In addition to pattern characterization for movement, an embodiment of the present disclosure also measures and uses information on holding a particular position for a length of time for pattern characterization. Information on maintaining a particular position can be helpful for assessing stress factors with a specific examination or type of examination.

Pattern recognition for movement assessment can use a number of movement analysis methods familiar to those skilled in human kinetics or kinesiology, such as methods commonly used in sports training and coaching and sports medicine. This includes effective use of principles of effective motor action and motor redundancy as they relate to the human anatomy.

### Sensor Types

The term “sensor” (as used in this disclosure) refers to various devices which can be used for the Ultrasound Ergonomic metrics. Indeed, in any one particular embodiment, one or more of any type of sensor 30 can be employed. The variety of potential usable sensor devices includes, but is not limited to:

(i) Accelerometer: measure body movement to track movement from one position to another.

(ii) Gyroscope: measures rotation for a variety of purposes and can be used for tracking arm or joint rotation. For example, a gyroscope can sense when a wristwatch is turned for a look at the watchface, energizing the display accordingly. (iii) Magnetometer: used for improved accuracy in motion tracking.

(iv) Ambient temperature sensor. Another sensor that could be used in algorithms that report bio-data. For example, ambient temperature could be compared to skin temperature in the service of determining exertion levels;

(v) Skin temperature sensor. Comparison of skin temperature to ambient temperature provides a useful measure of exertion.

(vi) Weight and pressure sensors.

(vii) Contact and proximity sensors.

Reference is made to sensors for biometrics, such as described in the following references, which are incorporated herein in their entirety:

2013 IEEE International Conference on Robotics and Automation (ICRA) Karlsruhe, Germany, May 6-10, 2013. “Soft Wearable Motion Sensing Suit for Lower Limb Biomechanics Measurements” Yigit Menguc, Yong-Lae Park, Ernesto Martinez-Villalpando, Patrick Aubin, Miriam Zisook, Leia Stirling, Robert J. Wood, Conor J. Walsh.

© XSENS TECHNOLOGIES—VERSION Apr. 3, 2013 1. Xsens MVN: Full 6DOF Human Motion Tracking Using Miniature Inertial Sensors, Daniel Roetenberg, Henk Luinge, and Per Slycke.

Second Skin Captures Motion. MIT Technology Review—Applied use of photosensors. Graduate student Dennis Miaw demonstrates Second Skin. The system tracks arm motion and vibrates to indicate how to correct position.

Reference is also made to the following: U.S. Pat. No. 8,021,312 (Kinnunen); U.S. Pat. No. 8,162,857 (Lanfermann) U.S. Pat. No. 8,527,028 (Kurzweil); U.S. Pat. No. 8,560,044 (Kurzweil); U.S. Pat. No. 8,684,924 (Ouwerkerk); US 2009/0299232 (Lanfermann); US 2014/0070957 (Longinotti-Buitoni); US 2014/0228649 (Rayner); and US 2014/0142459 (Jayalth); all of which are incorporated herein by reference in their entirety.

### Sensor Coupling

Sensors 30 can be coupled to the sonographer in a number of ways, such as by attachment to the skin of the sonographer, by attachment to or integration with clothing such as shirt or lab-coat worn by the sonographer, or by attachment to one or more harnesses, armbands, wristbands, or other devices worn or carried by the sonographer. The application of the sensor to clothing might be removably accomplished (so that the sensor can be removed and re-used) or the device might be non-removably applied (so that the sensor is secure and unlikely to interfere with the work activities). The sensor 30 may be integrated within the clothing textile so that it cannot be removed or disabled or so that it is otherwise difficult to detect.

Sensor 30 can be applied solely on the skin or clothing, or on both the skin and clothing. The sensor is preferably applied at the skin/clothing location which is appropriate for tracking a particular MSD/disorder. Accordingly, for example, to properly track the hand movement of the sonographer, it is preferable to monitor a sensor applied directly to, or very near, the hand, rather than monitor a sensor applied on a shirtsleeve or shoulder.

Embodiments include use of sensors of different types, coupled differently to the sonographer. For example, a first sensor can be applied onto the skin and a second and third sensor can be applied to clothing or lab coat.

A plurality of coupled sensors can be employed, and can be applied solely on the skin/clothing, or on both the skin/clothing. For example, a first sensor can be applied on the skin and a second and third sensor can be applied to the clothing.

As noted previously with respect to FIG. 2C, sensors can also be coupled to supporting furniture and equipment used by the sonographer. This can include sensors 30 on chair 40 or other seating as well as sensors installed along the frame or other portion of ultrasound apparatus 10 itself.

### Feedback to the Sonographer

Feedback/data/biometrics from each sensor can be immediately provided to the sonographer so that the sonographer is aware of potential strain on the body that may result in injury. The feedback can be provided visually, for example, using the ultrasound system's display or using a personal monitor/device.

Alert signals can be used to alert the sonographer of good practices and/or non-preferred/undesired/strain. Such alerts can be provided to the sonographer in various ways, for example:

(i) visual or audible alert, using the ultrasound system's display;

(ii) visual or audible alert, using a personal device, such as a wrist bracelet/monitor. This wrist device could be worn on the sonographer's wrist/pocket, for example; or

(iii) visual or audible indication using a device pinned/attached to the sonographer's clothing or located within a pocket.

The visual alert can be, for example, a flashing bright light or a particular color of light. The audible alert can be, for example, a particular bell, whistle, sound, vibration or song. The signals can be personalized to a particular individual, based on personal preference.

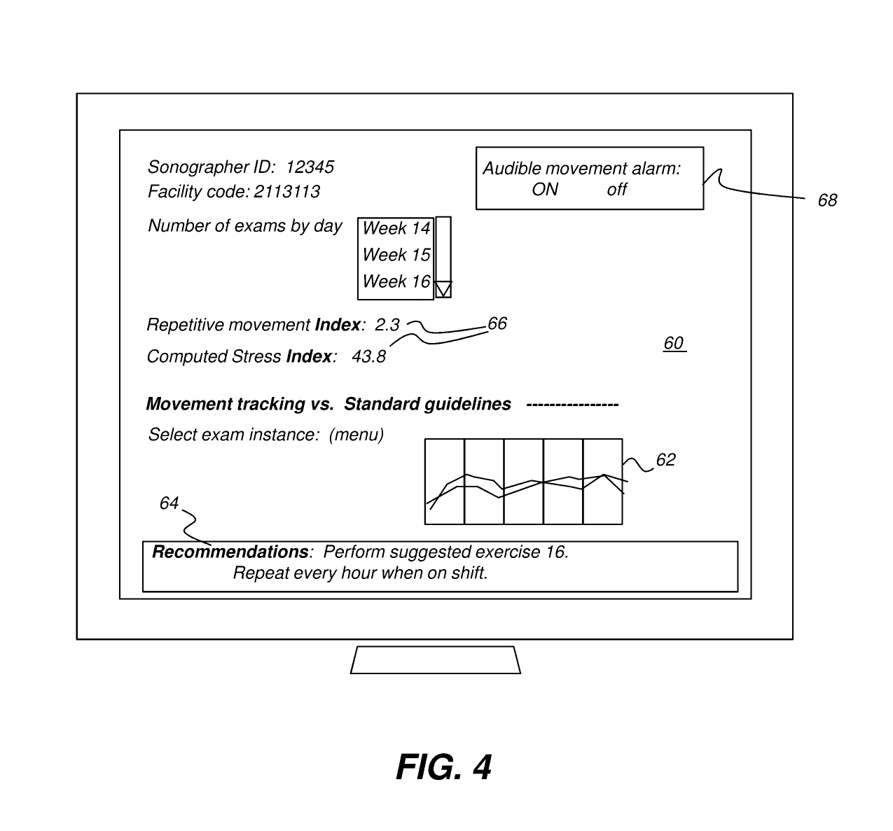

Graphical utilities can also be provided for feedback. Referring to the exemplary display screen of FIG. 4, there is shown a graphical report 60 that can display to the sonographer, showing current settings 68 that have been entered as well as cumulative data, such as biometric data gathered for a single exam or for a designated hourly period, for a work-shift, for a week, month, or other time period. Data displayed can include number of exams, differentiated by type, statistics on movement patterns deviating from established norms, indices 66 that characterize overall posture, incidents or motions that may cause discomfort, stiffness, or pain due to awkward positioning, extension, or duration fixed in a single position, for example. Other indices that can be calculated and used by the sonographer for comparison and for possible future improvement can include an overall stress index computed based on amount of time spent in reaching with the full arm or extending the arm above shoulder level, intervals between measurements, relative amount of required repositioning of monitor or other equipment between or during an exam, amount of time spent holding an awkward neck angle maintained for keeping the display screen in sight while applying the transducer against the patient, and other conditions. An index can be computed using averaging, mean-square-error estimation, or other standard statistical tool.

Metrics can be acquired for a particular individual or cumulative metrics and statistics obtained for all practitioners at a particular imaging site or unit. Time graphs 62 enable individuals, team members, training staff, or management to observe tendencies to improve over time, or to identify lapses into poor or careless behavior, or other trends exhibited by an individual or by members of the ultrasound staff. Graph 62 can display target values, for example, to provide a quick visual indication of sonographer movement patterns relative to a norm or desired goal.

According to an embodiment, the sonographer can generate and edit a feedback mode profile that dictates how the movement analysis utilities available from the ultrasound system make results known to the sonographer. Some practitioners may see intermittent beeping sounds, flashing lights, or other immediate feedback as undesirable or distracting during the exam. Sonographers may want to view long term data, such as at the end of the day or at a periodic review session, including sessions with multiple team members or staff for an ultrasound unit, for example.

According to an alternate embodiment, the display also offers helpful information with one or more messages 64 on potential ways to improve movement patterns. The display can also suggest activities, such as stretching or relaxation exercises, that can help the sonographer to reduce stress, relieve muscle tension, and possibly counteract at least some portion of the discomfort that may result from particular movement patterns that have been detected.

The feedback/data/biometrics would be relevant to the ergonomic aspect of the sonographer's work activities, so as to mitigate/reduce occupational injuries.

The method of the present disclosure can also provide a computer storage product having at least one computer storage medium having instructions stored therein causing one or more computers to perform the described calculations and to display features.

Consistent with one embodiment, the present invention utilizes a computer program with stored instructions that control system functions for sensor data acquisition and processing. As can be appreciated by those skilled in the data processing arts, a computer program of an embodiment of the present invention can be utilized by a suitable, general-purpose computer system, such as a personal computer or workstation that acts as an image processor, when provided with a suitable software program so that the processor operates to acquire, process, transmit, store, and display data as described herein. Many other types of computer systems architectures can be used to execute the computer program of the present invention, including an arrangement of networked processors, for example.

The computer program for performing the method of the present invention may be stored in a computer readable storage medium. This medium may comprise, for example; magnetic storage media such as a magnetic disk such as a hard drive or removable device or magnetic tape; optical storage media such as an optical disc, optical tape, or machine readable optical encoding; solid state electronic storage devices such as random access memory (RAM), or read only memory (ROM); or any other physical device or medium employed to store a computer program. The computer program for performing the method of the present invention may also be stored on computer readable storage medium that is connected to the image processor by way of the internet or other network or communication medium. Those skilled in the image data processing arts will further readily recognize that the equivalent of such a computer program product may also be constructed in hardware.

It is noted that the term “memory”, equivalent to “computer-accessible memory” in the context of the present disclosure, can refer to any type of temporary or more enduring data storage workspace used for storing and operating upon image data and accessible to a computer system, including a database. The memory could be non-volatile, using, for example, a long-term storage medium such as magnetic or optical storage. Alternately, the memory could be of a more volatile nature, using an electronic circuit, such as random-access memory (RAM) that is used as a temporary buffer or workspace by a microprocessor or other control logic processor device. Display data, for example, is typically stored in a temporary storage buffer that is directly associated with a display device and is periodically refreshed as needed in order to provide displayed data. This temporary storage buffer can also be considered to be a memory, as the term is used in the present disclosure. Memory is also used as the data workspace for executing and storing intermediate and final results of calculations and other processing. Computer-accessible memory can be volatile, non-volatile, or a hybrid combination of volatile and non-volatile types.

It is understood that the computer program product of the present invention may make use of various data manipulation algorithms and processes that are well known. It will be further understood that the computer program product embodiment of the present invention may embody algorithms and processes not specifically shown or described herein that are useful for implementation. Such algorithms and processes may include conventional utilities that are within the ordinary skill of the sensor and signal processing arts. Additional aspects of such algorithms and systems, and hardware and/or software for producing and otherwise processing the acquired data or co-operating with the computer program product of the present invention, are not specifically shown or described herein and may be selected from such algorithms, systems, hardware, components and elements known in the art.

The invention has been described in detail, and may have been described with particular reference to a suitable or presently preferred embodiment, but it will be understood that variations and modifications can be effected within the spirit and scope of the invention. The presently disclosed embodiments are therefore considered in all respects to be illustrative and not restrictive. The scope of the invention is indicated by the appended claims, and all changes that come within the meaning and range of equivalents thereof are intended to be embraced therein.

What is claimed is:

1. A method comprising:
recording a biometric signal received from a sensor that is coupled to the skin, clothing, or seating of a sonographer;
identifying a pattern from the recorded signal, wherein the pattern relates to sonographer activity for an ultrasound exam; and
providing feedback information about the sonographer activity in response to an analysis of the identified pattern.

2. The method of claim 1 wherein the biometric signal indicates sonographer movement.

3. The method of claim 1 wherein the biometric signal indicates a body temperature.

4. The method of claim 1 wherein the sensor is taken from the group consisting of an accelerometer, a gyroscope, and a magnetometer.

5. The method of claim 1 wherein the analysis comprises comparing the identified pattern with a stored pattern.

6. The method of claim 1 wherein providing feedback comprises providing audible feedback.

7. The method of claim 1 wherein the analysis of the identified pattern relates to size characteristics of a patient.

8. The method of claim 1 wherein the analysis of the identified pattern relates to size, age, or weight characteristics of the sonographer.

9. The method of claim 1 wherein the signal is a first signal and wherein the sensor is a first sensor and further comprising recording a second biometric signal from a second sensor that is coupled to the skin or clothing of the sonographer and wherein identifying the pattern uses both the first and second signals.

10. The method of claim 1 wherein the identified pattern indicates extension of a limb of the sonographer.

11. A method comprising:
coupling at least one biometric sensor to the skin or clothing of a sonographer;
monitoring a signal from the at least one coupled biometric sensor relating to sonographer movement during an ultrasound examination; and
providing feedback about sonographer activity to the sonographer according to the monitored signal from the at least one sensor.

12. A method, comprising:
attaching at least one sensor to the skin, clothing, or seating of a sonographer;
monitoring a characteristic of the at least one sensor as the sonographer conducts work activities; and
providing feedback to the sonographer regarding the conducted work activities based on the monitored characteristic of the at least one sensor.

13. The method of claim 12 wherein the feedback is provided visually or audibly.

14. The method of claim 12 wherein the feedback is transmitted or stored to a computer.

15. The method of claim 12 wherein a plurality of sensors are applied to the skin or clothing.

16. The method of claim 12 wherein the monitored characteristic relates to sonographer posture.

17. The method of claim 12 wherein the monitored characteristic relates to sonographer motion patterns.

18. The method of claim 12 wherein the monitored characteristic relates to sonographer exertion.

19. The method of claim 12 wherein the monitored characteristic is indicative of Musculoskeletal Disorder.

20. The method of claim 12 wherein providing feedback comprises using a feedback mode profile associated with the sonographer.
